(19) United States
(12) Patent Application Publication
(10) Pub. No.: US 2011/0300245 A1
(43) Pub. Date: Dec. 8, 2011

(54) Topical, palatable, anti-inflammatory and analgesic for specific application to intraoral, gingival and extraoral areas correlated with, and related to, the trigeminal nerve and its nerve branches and plexus. To mitigate, attenuate or prevent migraine-type headache, cluster-type headache, tension-type headache, post-traumatic headache, atypical facial pain and cervical muscle spasm as well as other idiopathic head or facial pain or discomfort, associated with, or exacerbated by, inflammation

(76) Inventor: Wendy Lou Marshall, Yarmouth Port, MA (US)

(21) Appl. No.: 12/792,950

(22) Filed: Jun. 3, 2010

(51) Int. Cl. A61K 36/886; A61K 38/14; A61P 29/00; A61K 31/7048; A61K 36/00; A61K 38/22; A61K 31/7004

(52) U.S. Cl. 424/744; 514/12.2; 514/20.9; 514/23; 514/27; 424/725

(57) ABSTRACT

The invention is a new unique, palatable, topical, anti-inflammatory and analgesic designed around a specific set of painful conditions, with specifically designated applications, using a different methodology and formulation, with no NSAIDS, steroids or salicylites, than the prior art for this specific set of painful conditions. Thus creating an improved dissimilar system, preventing the prior arts required invasive, costly and inefficient medical procedures, and with a unique lack of negative effects when compared with the prior art. Thereby, facilitating an anti-inflammatory analgesic effect to areas correlating or relating to the trigeminal nervous system, both intraoral and extraoral. Trigeminal tenderness is noted consistently in many painful conditions and this invention can be used to mitigate these areas of tenderness and inflammation preventing or relieving, migraine, cluster-type headaches, tension-type headache, post-traumatic headache, atypical facial pain and cervical muscle spasm as well as other related pain or discomfort exacerbated by trigeminal inflammation.

The invention relates to a method for mitigation of migraine, cluster-type headaches, tension-type headache, post-traumatic headache, atypical facial pain and cervical muscle spasm. More particularly the invention relates to a new method of treatment which is, non-invasive, non-sedating and non-toxic and requires no special equipment. The method of the invention comprises delivering a topical anti-inflammatory composition, applied by the human finger, or by any such means as the user might prefer, which composition and method does not include NSAIDS, salicylites or glucocorticoid steroids, to a specific intraoral area of tenderness consistently noted in persons with headache that appears closely associated with several painful conditions: migraine, cluster-type headache, tension-type headache, post-traumatic headache, atypical facial pain, cervical-muscle spasm and the facial trigeminal nerve areas as specifically outlined in the abstract and claim afore-described, as well as inflammation upon a distinct area of maxillary alveolar tenderness that is consistently noted in patients with headache, even in the headache-free state. Symptomatic mitigation of head or facial pain can be obtained by applying the invention to specific trigeminal nerve areas correlating to the intraoral area of the inferior alveolar branch along the lower jaws outer gingival tissue between teeth and gums about the area of the rearmost two molars and the wisdom tooth, and extraoral correlating with the: auriculotemporal nerve at the front base of the ear, temporomalar nerve at both jaw and temple, supratrocheal, lacrimal and supraorbital nerves of the forehead, buccal nerve of the cheek, mental nerve of the lower jaw, infratrochlear and nasal nerves along the nose, and infraorbital nerve of the cheek area adjacent the nose and under the eye. The invention provides anti-inflammatory and analgesic action to the largest number of areas which could be described as correlating or relating to the entire trigeminal plexus and structures which are correlated with pain in the head, face or neck. The composition comprises at least one, or any combination of, anti-inflammatory, herbs and/or botanicals, or any singular or any mixture of any grouping of herbs and/or botanicals, (initially boswellia/boswellic acid), in the form of a liquid, ointment, oil, acid extract, ground leaf, root or stem, cream, lotion, gel, powder, paste, solid or semi-solid, dissolved, distributed or dispersed in dimethyl sulfoxide in a liquid, gel or solid or semi-solid form between 0.01% and 99.99% concentration, (initially 50%), mixed with appropriate water, urea or ethanol, said urea or ethanol and NaCI or KCI buffering the side effects of the 50% dimethyl sulfoxide, the liquids, acids, oils, emulsifiers, stabilizers and/or thickeners (initially carbopol) to create an invention allowing adherence to gingival, head and neck tissues for the purpose of anti-inflammatory and analgesic action, with a significant mitigation of known unpleasant effects of dimethyl sulfoxide such as a mild tingling sensation when applied to the skin, dry skin, malodorous emanations and the resulting foul body odors upon application, by the addition of urea or ethanol, made palatable and having a pleasant aroma by mixing with mentha x piperita and a sweetener (initially stevia) for both palatability and further amelioration of the very foul taste of, and malodorous conditions flowing naturally from dimethyl sulfoxide before and, also, after application.

### PRIOR ART

Until I created my invention, the prior art to achieve an approximately similar effect upon some intraoral trigeminal nerve areas for the purposes and claims stated herein was limited to exclusively intraoral application to a specific trigeminal point, and even then required invasive office procedures, multiple physicians visits, extended time allowances, and extensive costs and travel; to utilize an intraoral cold laser or cooling machine and then to follow those invasive, inconvenient and costly procedures with a daily application of a costly topical paste which was only available through a compounding pharmacy, into which an NSAID, salicylites or glucocorticoid steroid was mixed for intraoral topical daily application [23] [26] [29] [30]. None of these prior methodologies, or prior active ingredients, is any part of my invention. My invention does not use any cooling device or laser, or any NSAIDS, salicylites or glucocorticoid steroids. My invention is the first to make the foul-tasting substance dimethyl sulfoxide more palatable as a specific stated goal of this invention and claim. There is no travel; no high costs, no limited availability, no need for a compounding pharmacy to obtain the NSAID, salicylites or steroid pastes or special medical settings or medical visits required and virtually no time lost employing my invention. My invention addresses the same painful inflammation, and associated pain, which are correlated with tissues associated with, or in relation to, the trigeminal nerve structures/plexus and surrounding tissues, but by a completely different methodology, approach, formula and substances.

### BACKGROUND OF THE INVENTION

General:

The pathophysiology of migraine is controversial, but the prevailing theory describes a persistent neurogenic dural inflammation with plasma extravasation (swelling) (see [35] and [49]). Friedman (e.g., [32]) found that this neurogenic inflammation may also occur locally, closely adjacent to a relatively accessible maxillary nerve segment.

Friedman identified a distinct area of maxillary alveolar tenderness that is consistently noted in patients with headache: vascular, tension-type, post-traumatic, even in the headache-free state (asymptomatic patients). This area has consistently been identified in patients suffering from facial pain [18] or posterior cervical muscle hyperactivity (spasm) [21]. This zone of tenderness, usually absent or minimal in pain free individuals [18], occurs in the maxillary third molar apical area even if edentulous, and correlates closely with a plexus formed by the posterior superior alveolar branch of the maxillary nerve. It is also noted in patients with atypical facial pain [17] [18] and cervical muscle spasm [21].

In preliminary data analysis, 1026/1100 (93.2%) mostly asymptomatic migraine and tension-type headache patients exhibited maxillary alveolar tenderness, with laterality and degree of tenderness closely related to laterality and severity of symptoms. This consistent finding has been corroborated by several neurologists [34].

To identify the maxillary alveolar tenderness (MAT), the examiner's index finger is pressed upward and backward as far as possible along the vestibule forming the roof of the cheek pouch when the mouth is partially opened - when opened fully, muscular contraction reduces space for the examination. If necessary for clearance, individuals are instructed to move the mandible laterally to the side being examined. Palpation is performed from the apical area of the maxillary third molar (whether the tooth is present or not) forward as far as the cuspid region. The MAT does not extend this far anteriorly, but tenderness can be better appreciated when compared with the adjacent nonsensitive gingiva [21].

The MAT is described by some dentists and chiropractors as lateral pterygoid muscle hyperactivity (spasm). However, this main jaw opening muscle lies far from the surface and therefore is not accessible to palpatio [41] [16]. It can, however, be assessed indirectly; isometric force application to this muscle elicits pain if spasm is present; the patient resists a strong closing (upward) force applied to the chin [15]. On over 2,500 patients examined in this manner, no relationship was observed between lateral pterygoid muscle spasm and MAT. Chiropractors occasionally report relief from MAT palpation—local edema is dispersed, reducing pressure against the maxillary nerve [34].

In a pilot study of thirty asymptomatic migraine patients with a unilateral history, blinded, inexperienced examiners selected the symptomatic side in 27/30 (90%) patients, based on the laterality of intraoral palpation findings. In a study of patients with cervical muscle spasm and pain, MAT was palpated in 623 out of 663 cases (93.9%) [21]. It was also noted that in severe trauma cases with major cervical injury, MAT is severe and cannot be eliminated, even by instillation of local anesthetic to the area.

In 18 consecutive patients with atypical facial pain [24] (recently reclassified as facial pain, (International Headache Society)), ipsilateral tenderness and increased temperature were found in 15 and 17 patients, respectively; in two control groups, often patients, no significant association was observed between the area associated with maxillary tenderness and increased temperature. A YSI (Yellow Springs International) Model 43TA tele-thermometer covered by a disposable plastic sheath was used bilaterally to record temperatures [18].

A similar experiment (intraoral palpation, temperature recording) was performed on 40 patients during unilateral episodic migraine or tension-type headache in the departments of Neurology, Dentistry, Emergency Medicine (Westchester Medical Center, Valhalla, N.Y.) a private practice limited to oromandibular dysfunction (MHF, Mt. Vernon, N.Y.), and Our Lady of Mercy Hospital (Bronx, N.Y.). The posterior maxillary molar periapical areas were palpated bilaterally for tenderness, and their temperatures were recorded, as in the previously described facial pain experiment [18]. During unilateral migraine or tension-type headache, the upper third molar areas (even if the teeth were missing) were palpated on both sides for tenderness. Furthermore, the temperature was consistently higher (37 of 40) and the area more tender (39 of 40) on the ipsilateral side [24]. Tenderness and elevated temperature are signs of inflammation [34].

Intraoral Chilling:

Friedman developed an apparatus [20] [22] [23] [33] to resolve edema by local vasoconstriction. Chilling of the edematous MAT area decreases intracapillary pressure by reducing local blood flow; edema (excessive interstitial fluid) is encouraged to return to the micro circulation via the capillary wall, reducing pressure against the maxillary nerve. The device consists of hollow metal tubes shaped to fit over the MAT. The metal is covered by a disposable plastic sheath. The tubes are connected to an ice water source by rubber tubing. The ice water, driven by a small pump, passes through and chills the metal, then flows to an identical arrangement on the opposite side, and back to its source [21]. Even if symptoms are unilateral, the device is applied bilaterally because unilateral application occasionally caused a symptom (and MAT) side-shift. In all applications, the device is held by the patient—usually for 37 minutes—based on extensive symptomatic patient applications using varying times [34]. In over 4,000 applications in two private clinics, a tertiary medical center emergency department, and a department of medicine out-patient controlled study, side effects have not been observed [34]. The device has received FDA approval for migraine and muscle spasm, and has been declared a non-significant risk by the New York Medical College and the Westchester Medical Center Institutional Review Boards (IRBs). Because the procedure involves local intraoral vasoconstriction, it is referred to as IVC, and the device is called the IVC device [34].

In a study conducted at Westchester Medical Center, IVC was applied to 25 patients with severe enough migraine or tension-type headaches (average pretreatment headache duration over 30 hours) to require an Emergency room visit. In 20 of the patients (80%), including three pregnant women, symptoms were relieved within 40 minutes, solely by IVC. The mean migraine score (0-10 scale) went from 7.74 to 3.7 and the mean TTH score from 7.8 to 2.7. No side effects were reported and the patients remained comfortable the following day, with no rebound headaches [25].

Additionally, thirty-five patients were treated in the outpatient department of the New York Medical College Department of Medicine and a faculty practice at St. Agnes Hospital, during severe migraine (average pretreatment duration 17+ hours). In randomized fashion, the patients were treated by 40 minutes of IVC, 50 mg of oral sumatriptan, or (placebo) tongue chilling (40 minutes), using the same device. Significant mean headache relief was obtained by IVC and sumatriptan at 1, 2, 4, and 24 hours after the initiation of treatment, with poor relief obtained by placebo (0-10 scale). IVC was more effective than sumatriptan at all four time intervals. Significant nausea relief was obtained by maxillary chilling and sumatriptan at the same post treatment time intervals. At 24 hours, some headache and nausea recurrence was noted with sumatriptan but not with IVC [28].

Persistent neurogenic inflammation in the maxillary nerve area appears to mediate not only headache but cervical muscle spasm as well. This observation is not surprising as the head and neck operate as a single functional unit [21] [27]. Over 100 years ago, Sherrington [59] reported that in the decerebrate cat, excitation of a portion of the trigeminal nerve is enough to cause relaxation of the rigid neck muscles. Since then, experiments have confirmed a close trigemino-cervical relationship [43] [1]. Because the head and neck are one functional unit, cervical musculoskeletal disorders can refer as headache, jaw, or facial pain [14].

A non-blinded study was performed on 12 subjects with clinically determined cervical muscle spasm (hyperactivity). All subjects reported neck pain at rest and during cervical range of motion (ROM), and six reported headache. MAT was present in all cases. Upper trapezius surface electromyography (EMG) was performed before and after relatively brief IVC. Following treatment, 9 out of 12 subjects demonstrated a reduction of EMG activity. In these same nine subjects, there was a mean increase of 10.58 degrees plus or minus 6.95 degrees of motion in the single most painful cervical spine movement. Resting neck pain was relieved or eliminated in 8 out of 12 subjects, and four out of six accompanying headaches were also relieved, from only 15 minutes of IVC [21]. Since that time, IVC application has been extended to 37 minutes. On over 4,000 applications, an 80% success rate has been recorded (significant improvement immediately after treatment), with no side effects [21]. In headache, MAT may well be the origin of the problem. In cervical muscle spasm, the dysfunction appears to create the MAT, further perpetuating cervical muscle spasm. This type of loop is not unique. For example, lactic acid is created from muscle contraction and further exacerbates the muscle pain and soreness. IVC interrupts the loop. In some cases, this treatment replaces cervical physical therapy or chiropractic. More often, it is used as an adjunct to ongoing cervical treatment. It has been found to work especially well in headaches coexisting with cervical muscle spasm [34].

Low-level (non-cutting) Lasers:

Cryotherapy (IVC) accesses the trigeminal system intraorally, where it is unprotected by skin or bone, which may be a factor in the dramatic results obtained. Evoked potentials (EPs), the electric responses of the nervous system to various stimuli, induce a cortical response, the amplitude of which increases linearly with pain intensity and decreases when pain is attenuated [3].

In pharmaceutical research, EP amplitudes are commonly used to evaluate drugs because the placebo effect is eliminated. Low-level (non-cutting) lasers also produce a local anti-inflammatory effect. Based on this, Friedman developed several laser devices [19] [30].

In a trigeminal somatosensory (as opposed to visual or auditory), placebo-controlled EP study, the infraorbital foramen was used as the electrical input. After electrical input at the left infraorbital foramen, on 24 experimentally blinded pain-free subjects, Helium Neon laser irradiation (1. mW, 632.5 nm, 50 Hz) was performed for 2 minutes on 12 of these subjects, and sham irradiation on the other 12, at the left maxillary third molar apical area. Four far field step latencies and amplitudes were recorded at base line, immediately after intervention, and 10 and 20 minutes after intervention. Laser irradiation was performed on the same intraoral area used for headache treatment. In the irradiated group, an immediate (average) somatosensory trigeminal evoked potential (STEP) amplitude decrease from base-line of 60% occurred, with further reduction to 65% and 72%, at the 10- and 20-minute intervals. No significant change occurred in the sham irradiation group [51].

Other studies have also suggested that a strong connection exists between trigeminal and cervical motor and sensory responses. These experiments suggest that the intraoral marker, (area of maxillary alveolar tenderness), for the above pain disorders, is a local inflammation, associated with increased local blood flow (vasodilation) and edema. The increased local temperature and tenderness are classic signs of inflammation, and the significant response to local cooling is caused by edema (swelling, another classical sign of inflammation) resolution.

Topical Anti-inflammatory Gel:

If secondary edema from a local inflammation causes migraine and TTH, and responds to local anti-inflammatory treatment (chilling), topical NSAID application to the same area should also aid in treatment of these headaches.

Friedman developed such a topical anti-inflammatory gel (Ketoprofen) [26] [31] [29]. It was applied by patients to the same intra-oral area for headache prevention. Twenty episodic migraine, TTH, and posttraumatic headache patients were enrolled in an open-label headache prevention study [31]. Patients kept a headache diary for 60 days, recording headache type, frequency, severity (1-10 scale), duration (total monthly headache hours), headache medications analgesics taken, and side effects. During the second 30 days, medication was applied. Ketoprofen was compounded with organogel, serving as the vehicle to deliver the drug to the tissue. Patients placed a disposable cheek shield between the molar teeth and the cheek; patients then applied the gel to the tissue bilaterally, using a cotton applicator. This procedure was followed once daily. If symptoms were exclusively unilateral, the medication was applied to the symptomatic side only. Headache burden was defined as the average intensity of each headache (0-10 scale) multiplied by its duration in hours. The average monthly headache burden score for the 20 patients enrolled in the study went from 454.8 (30-day baseline) to 86.5 p<0.001 during the 30-day treatment phase. Additionally, analgesic and headache medication intake was significantly reduced from baseline during the treatment phase, and side effects were minimal. Usually, migraine preventive medications must be taken for at least a few months before effectiveness can be determined and are rarely prescribed for TTH [51]. In practice, because the gel is now more adhesive, the cheek shield is rarely used [34].

Dimethyl Sulfoxide (DMSO):

Jacob [40] was the first to recognize the medical applications of dimethyl sulfoxide (DMSO). DMSO penetrates the skin quickly [44] and was initially shown to act as an anti-inflammatory and diuertic agent with a resulting reduction of edema in animals (e.g., [13], [36] and [64]). This was later shown to possess lifesaving potential in stroke and head patients [9]. DMSO reduces inflammation by several mechanisms. It is an antioxidant, a scavenger of the free radicals that gather at the site of injury [39]. Furthermore, laboratory studies suggest that DMSO cuts pain by blocking peripheral nerve C fibers [12].

Numerous studies have successfully applied the anti-inflammatory effects of DMSO to treat musculoskeletal problems. Demos et al. [10] analyzed the findings of a 1964 pilot clinical pharmacology study into the safety and effectiveness of DMSO in treating musculoskeletal and dermatologic diseases by The Squibb Institute for Medical Research and 76 subsequent clinical investigations in the United States. They found that 80% of the acute patients and 60% of chronic patients showed improvement when treated with DMSO.

In a clinical study of 103 patients with acute and chronic musculoskeletal injuries and inflammations [47], the application of DMSO resulted in pain relief of varying degrees in most patients. Spectacular symptomatic relief occurred in all six patients with acute calcified supraspinatus bursitis. Within 10 minutes of application of DMSO 1.0 ml the other five patients with acute supraspinatus tendon pain associated with calcification experienced pain relief. After continued applications every four hours over the following 24 hour period, complete recovery was experienced with normal use of the shoulder. In 27 patients with rheumatoid arthritis of varying degrees of acuteness and severity, all but four had some degree of relief of pain following the application of DMSO.

A multi-clinic trial of DMSO on patients of rheumatoid arthritis was performed by the Committee on Clinical Drug Trials of the Japanese Rheumatism Association during April to October 1965 [48]. It was found that DMSO exhibited analgesic and anti-inflammatory effects, relieving joint pain and increasing the range of joint motion and grip strength.

In 1972 the National Academy of Sciences evaluated the scientific data on DMSO and concluded it was at least as effective as currently approved treatments for three musculoskeletal inflammatory problems in man.

Stewart et al. [61] were the first to publish a report on intravesical dimethyl sulfoxide (DMSO) in the treatment of interstitial cystitis. Since then, other investigators have confirmed its therapeutic value (e.g., [62]). Intravesical dimethyl sulfoxide (DMSO) was used in the treatment of 213 patients with various inflammatory conditions involving the lower genitourinary tract, including intractable interstitial cystitis, radiation cystitis, chronic prostatitis, and chronic female trigonitis [60]. Significant symptomatic relief was achieved in the majority of patients so treated, and no systemic or local toxicity was noted.

Ulcerative colitis is a diffuse inflammation of the mucous membranes of the colon, which when severe, leads to extensive ulceration. Salim [57] conducted a double-blind, randomized study investigating the role of oxygen-derived free radical scavengers in the management of recurrent attacks of ulcerative colitis. To this end, allopurinol (50 mg four times a day) and dimethyl sulfoxide (500 mg four times a day) were administered orally. Patients with recurrent attacks of moderate proctosigmoidal ulcerative colitis, in spite of prophylaxis with orally administered sulfasalazine (2 gm daily), were given 10 mg prodnisolone by mouth four times a day: 500 mg sulfasolazine by mouth four times a day; and morning and evening retention steroid enema (Prodsol, 20 mg) alone or with allopurinol or dimethyl sulfoxide. After 2 weeks of treatment with sulfasalazine and prodnisolone alone, 51% of patients (n=45) were free of symptoms. Addition of allopurinol (n=46) or dimethyl sulfoxide (n=45) to the mentioned regimen controlled the symptoms within 2 weeks in 84% of patients (p<0.01). During 12 months of prophylactic treatment, 5% of patients (n=42) who were given sulfasolazine (2 gm daily) and allopurinol and 5% of patients (n=40) who were given sulfasalazine (2 gm daily) and dimethyl sulfoxide relapsed compared with 25% of patients who were given sulfasoizine (2 gm daily) alone (p<0.05).

Karaca et al. [42] found that intravenous DMSO can rapidly reduce elevated intracranial pressure (ICP) in severe closed-head injury patients and that it improves neurological outcome. Here, ten patients with closed head trauma and elevated ICP ranging from 40-127 mm Hg were treated with intravenous dimethyl sulphoxide (DMSO) every 6 h for 1-10 days. Four patients received DMSO and intermittent oxygen. All patients showed a reduction of ICP after 24h and 7 had normal ICP after 6 days of treatment.

Jacob said at a hearing of the U.S. Senate Subcommittee on Health in 1980, “DMSO is one of the few agents in which effectiveness can be demonstrated before the eyes of the observers, if we have patients appear before the Committee with edematous sprained ankles, the application of DMSO would be followed by objective diminution of swelling within an hour. No other therapeutic modality will do this” [50].

Aloe Vera:

The anti-inflammatory qualities of aloe vera have been tested extensively. Aloe constituents with anti-inflammatory activity include: mannose-6-phosphate [7] [65]; the glycoproteins aloctin A and alprogen [58] [56]; a C-glucosyl chromone [38]; the anthraquinones [5]; and gibberellin [4].

Aloe vera gel taken orally helped reduce symptoms of patients with ulcerative colitis due to its anti-inflammatory effect [45]. Aloe vera has been shown to improve wound healing and inhibit inflammation, appearing to increase wound tensile strength [8], possibly due to the fibroblast stimulating activity of mannose-6-phosphate [7]. Furthermore, Reuter et al. [54] found that aloe vera gel significantly reduced UV-induced erythema after 48 hours, being superior to 1% hydrocortisone in placebo gel. Aloe also has antithromboxane activity, yet it maintains prostaglandin ratio without causing injured blood vessels to collapse. In an ear swelling assay, aloe vera extracts decreased inflammation by 29.2% when applied topically [6]. In another study, aloe vera appeared beneficial in an acute inflammatory model involving rats [66].

Acemannan, the major carbohydrate fraction in the gel, is a water-soluble long chain mannose polymer, which has been found in vitro and in animal studies to modulate immune function (particularly macrophage activation and cytokine production) and to accelerate wound healing. The macrophage stimulating principle of acemannan appears to reside in the high molecular weight polysaccharide Aloeride [53]. Acemannan has also been reported to exhibit antineoplastic and antiviral effects in vitro.

Other constituents include bradykininase, which possesses anti-inflammatory properties and magnesium lactate, which has antipruritic effects [69]. A mannose-rich polysaccharide fraction of aloe gel has been shown in mice to enhance antibody production [65]. Salicylic acid and other antiprostaglandin compounds may be responsible for aloe's local anti-inflammatory activity, possibly due to an inhibitory effect on the arachidonic acid pathway via cyclooxygenase [67].

Maloyl glucan compounds isolated from Aloe barbadensis Miller include 6-O-(1-L-maloyl)-alpha-, beta-D-Glcp (veracylglucan A), alpha-D-Glcp-(1-->4)-6-O-(1-L-maloyl)-alpha,-beta,-D-Glcp (veracylglucan B) and alpha-D-Glcp-(1-->4)-tetra-[6-O-(1-L-maloyl)-alpha-D-Glcp-(1-->4)]-6-O-(1-L-maloyl)-alpha,-beta-D-Glcp (veracylglucan C) [11]. Based on in vitro study, veracylglucan B demonstrated potent anti-inflammatory and anti-proliferative effects, while veracylglucan C exhibited significant cell proliferative and anti-inflammatory activities. Veracylglucan B and C appeared antagonistic and competitive in their effects on cell proliferation [ibid.].

### SUMMARY

It became apparent, upon studying the afore-described inflammation caused head, face and neck pain, and the state of the prior art for addressing it, that until I created my invention, the prior art to achieve an approximately similar effect upon some intraoral trigeminal nerve areas for the purposes and claims stated herein was exclusively intraoral, and even then limited to one area of focus, and even then required invasive office procedures, multiple physicians visits, extended time allowances, and extensive costs and travel; to utilize an intraoral cold laser or cooling machine and then to follow those invasive, inconvenient and costly procedures with a daily application of a costly topical paste or gel which was only available through a compounding pharmacy and was a paste, into which an NSAID or glucocorticoid steroid was mixed for intraoral topical daily application [23] [26] [29] [30]. While this was a legitimate prior alternative, it was only sparingly available and its primary, and as far as I am aware the only, full-practitioner is now deceased. None of these prior methodologies, or any prior active ingredients, are any part of my invention. My invention does not use any invasive cooling device, or any NSAIDS, salicylites or glucocorticoid steroids. My invention is the first to make a foul-tasting and malodorous substance, dimethyl sulfoxide, more palatable for intraoral use, as a specific stated goal of the invention. There is no travel; no high costs, no limited availability, no need for a compounding pharmacy to obtain any NSAID, salicylites or glucocorticoid steroid pastes or special medical settings or medical visits required and virtually no time lost employing my invention. My invention addresses the same painful inflammation, as well as other related points of pain and inflammation, which are correlated with tissues associated with or in relation to the trigeminal nerve structures/plexus and surrounding tissues, but by a completely different methodology, approach, formula and substances and resulted conditions and results, as well as the then state of the art for addressing them, that there was a different, and unique protocol by which to mitigate the afore-described head, neck and facial inflammation and resultant pain more easily, less expensively and more efficiently. It was this realization which then facilitated the creation of this invention in January 2009 and its subsequent submission for patent as it is unique.

### OBJECT OF THE INVENTION

The object of this invention is to reduce or eliminate intraoral and extraoral trigeminal correlated inflammation and associated pain localized in the maxillary third molar apical area, and the facial trigeminal areas afore-described and associated with the conditions listed above, by local topical anti-inflammatory and analgesic treatment without the use of NSAIDS, salicylites, glucocorticoid steroids, cold lasers or localized cooling but rather to achieve symptomatic and inflammation mitigation purely by the use of the inventions topical application, safely, inexpensively, easily and effectively. The invention is for long or short term use and with less potential for serious side effects such as mucous membrane erosion and general mouth and throat irritation from the action of NSAIDS and salicylites or the resulting large systemic load from NSAID or salicylites absorption, or the topical and systemic negative effects of glucocorticoid steroid use. The invention is also very efficient in mitigating the loss of productivity, time and efficiency caused by the debilitating pain of the afore-described conditions as these conditions inflict a dollar cost to society, as measured in 2004, of more than 24 billion¹. No doubt these costs have grown since that time. ¹ Thomson Medstat, a business of The Thomson Corporation (NYSE: TOC; TSX: TOC), conducted two studies analyzing the direct and indirect costs of migraine headaches among large U.S. employers. Sponsored by Ortho-McNeil Neurologics, Inc., the studies concluded that direct healthcare costs to American employers—including prescription drugs and in-patient, out-patient, and emergency room care—total approximately $12.7 billion annually. Indirect costs associated with migraine—including absenteeism, short-term disability, and workers' compensation—total $12 billion annually.

In accordance with the invention, the topical application of a combination of at least one member of anti-inflammatory herbs, spices or botanicals, dissolved, dispersed, mixed or distributed in a dimethyl sulfoxide carrier which uses urea or ethanol and a salt to mitigate unwanted side effects affects, odor, stinging upon application, foul body odors [37], and is flavored and sweetened for palatability and useful in delivering and enabling adherence of the invention to gingival areas of the mouth is formulated. The invention also embodies specific placement of this composition, an anti-inflammatory agent and carrier, to the periapical areas of the posterior molar teeth, the area of maxillary alveolar tenderness, in cases of migraine, cluster-type headaches, tension-type headache, post-traumatic headache, facial pain and cervical muscle spasm and to the facial trigeminal nerve and also to the head and facial trigeminal nerve areas specified as follows: correlating or relating to the intraoral area of the inferior alveolar branch along the lower jaws outer gingival tissue between teeth and cheek encompassing about the area of the rearmost two molars and the wisdom tooth, and extraoral correlating with the: auriculotemporal nerve at the front base of the ear, temporomalar nerve at both jaw and temple, supratrocheal, lacrimal and supraorbital nerves of the forehead, buccal nerve of the cheek, mental nerve of the lower jaw, infratrochlear and nasal nerves along the nose, and infraorbital nerve of the cheek area adjacent the nose and under the eye, as afore-described.

The invention can be applied in the form of liquids, ointments, creams, lotions, gels, solids, semi-solids, or pastes, with or without occlusion, by films or tapes or via specific adhesive bandages. The method of dispensing the composition is determined by the form of the carrier used for its presentation. As this will initially be a gel, a related form of appropriate application being best made by dispensing to the specific areas, via the human finger tip. Another advantage of absorption from the oral mucosal membrane is the elimination of the effect of food intake on the rate and on the resulting concentration of the invention during the absorption process. The exposure of the active agents to the low pH of the stomach is also avoided.

While I have described and given examples of preferred embodiments of my invention, it will be apparent to those well-acquainted in the art that revisions and modifications may be made without departing from my invention in its broader aspects. I therefore intend the appended claim to cover any and all such changes and modifications as fall within the true spirit and scope of my invention.

### REFERENCES

Abrahams, V. C. et al. (1975) Absence of monosynpatic reflex in dorsal neck muscle of the cat. Brain Research 92:130-131.

Becker, W. J. et al. (2006) Topiramate prophylaxis and response to triptan treatment for acute migraine. Headache 46:1424-1430.

Chen, A. C. N. et al. (1979) Brain evoked potentials are functional correlates of induced pain in man. Pain 6:365-374.

Davis, R. H. et al. (1989) Aloe vera and gibberellin. Anti-inflammatory activity in diabetes. Journal of the American Podiatric Medical Association 79(1):24-6.

Davis, R. H. et al. (1989) Anti-inflammatory activity of Aloe vera against a spectrum of irritants. Journal of the American Podiatric Medical Association 79(6):263-76.

Davis, R. H. et al. (1991) The isolation of an active inhibitory system from an extract of Aloe vera. Journal of the American Podiatric Medical Association 81:258-261.

Davis, R. H. et al. (1994) Anti-inflammatory and wound healing activity of a growth substance in aloe vera. Journal of the American Podiatric Medical Association 84(2):77-81.

Davis, R. H. et al. (1994)Aloe vera, hydrocortisone, and sterol influence on wound tensile strength and anti-inflammation. Journal of the American Podiatric Medical Association 84(12):614-619.

De la Torre, J. C. et al. (1973) Dimethyl sulfoxide in the treatment of experimental brain compression. Journal of Neurosurgery 38(3):345-354.

Demos, C H. et al. (1967) Dimethyl sulfoxide in musculoskeletal disorders. Annals of the New York Academy of Sciences 141:517-523.

Esau, M. F. and Rauwald, J. W. (2006) Novel bioactive maloyl glucans from aloe vera gel: isolation, structure elucidation and in vitro bioassays. Carbohydr Res 27;341(3):355-64. Epub 2005 Dec. 15.

Evans, M S. et al. (1993) Dimethyl sulfoxide (DMSO) blocks conduction in peripheral nerve C fibers: A possible mechanism of analgesia. Neuroscience Letters 150:145-148.

Formanek, K. and Kovacs, W. (1966) DMSO bei experimentellen Rattenpfotenodemen. In DMSO Symposium, Vienna, 1966 (G. Laudahn and K. Gertich, Eds.). pp18-24. Saladruck. Berlin.

Friedman, M. H. and Weisberg, J. (1982) Application of orthopedic principles in evaluation of the temporomandibular joint. Physical Therapy 62(5):597-603.

Friedman, M. H. and Weisberg, J. (1982) Screening procedures for temporomandibular joint dysfunction. American Family Physician 25:157-160.

Friedman, M. H. et al. (1983) Neglected conditions producing preauricular and referred pain. Journal of Neurology, Neurosurgery and Psychiatry 46:1067-1072.

Friedman, M. H. et al. (1994) Atypical facial pain: a localized maxillary nerve disorder? The American Journal of Pain Management 4:149-152.

Friedman, M. H. (1995) Atypical facial pain: the consistency of ipsilateral. Maxillary area tenderness and elevated temperature. The Journal of the American Dental Association 126:855-860.

Friedman, M. H. (1996) Treatment of vascular headache and atypical facial pain. U.S. Pat. No. 5,514,168.

Friedman, M. H. (1996) Treatment of vascular and tension headache atypical facial pain allergic rhinitis and cervical muscle hyperactivity. U.S. Pat. No. 5,527,351.

Friedman, M. H. and Nelson, A. J. (1996) Head and Neck Pain Review: Traditional and New Perspectives. The Journal of Orthopaedic and Sports Physical Therapy Volume 24(4), pp 268-278.

Friedman, M. H. (1997) Treatment of vascular and tension headache, atypical facial pain, and cervical muscle hyperactivity. U.S. Pat. No. 5,676,691.

Friedman, M. H. (1997) Treatment of vascular and tension headache atypical facial pain allergic rhinitis and cervical muscle hyperactivity. U.S. Pat. No. 5,693,077.

Friedman, M. H. et al. (1997) Ipsilateral intraoral tenderness and elevated temperature during unilateral headache. Headache 8(4):341-344.

Friedman M. H. et al. (1999) An alternative approach to acute headache treatment: an emergency department pilot study. Headache 10:131-134

Friedman, M. H. (2000) Intraoral topical anti-inflammatory treatment for relief of migraine, tension-type headache, post-traumatic headache facial pain, and cervical-muscle spasm. U.S. Pat. No. 6,139,861.

Friedman, M. H. and Weisberg, J. (2000) The craniocervical connection: a retrospective analysis of 300 whiplash patients with temporomandibular disorders. The Journal of Craniomandibular Practice 18(3):163-167.

Friedman, M. H. et al. (2001) Intraoral chilling versus oral sumatriptan for acute migraine. Heart Disease 3:357-61.

Friedman, M. H. (2002) Intraoral topical anti-inflammatory treatment for relief of migraine, tension-type headache, post-traumatic headache, facial pain, and cervical-muscle spasm. U.S. Pat. No. 6,423,697.

Friedman, M. H. (2002) Treatment of migraine, post-traumatic headache, tension-type headaches, atypical facial pain, cervical pain and muscle spasm. U.S. Pat. No. 6,450,170.

Friedman, M. H. et al. (2002) Intraoral topical non-steroidal anti-inflammatory drug application for headache prevention. Heart Disease 4:212-215.

Friedman, M. H. (2004) Local inflammation as a mediator of migraine and tension-type headache. Headache 44:767-771.

Friedman, M. H. and Casey, R. (2007) Method and device for treating migraine, tension-type and post-traumatic headache, atypical facial pain, and cervical muscle hyperactivity. U.S. Patent Application Publication US 2007/0032547.

Friedman, M. H. (2008) Headache An Intraoral Etiology and Noninvasive Treatment. In M. I. Weintraub, R. Mamtani and M. Micozzi (Eds) Complementary and Integrative Medicine in Pain Management, pp 125-135.

Goadsby, P. J. et al. (1990) Vasoactive peptide release in the extracerebral circulation of humans during migraine headache. Annals of Neurology 28:183-187.

Gorog, P. and Kovacs, I. B. (1969) Effect of dimethyl sulfoxide (DMSO) on various experimental cutaneous ractions. Pharmacology 2:313-319.

Herschler, R. J. (1981). Therapeutic dimethyl sulfoxide composition and methods of use. U.S. Pat. No. 4,296,104.

Hutter, J. A. et al., (1996) Antiinflammatory C-glucosyl chromone from Aloe barbadensis. Journal of Natural Products 59(5): 541-543.

Itoh, M. and Guth, P. (1985) Role of oxygen-derived free radicals in hemorrhagic shock-induced gastric lesions in the rat. Gastroenterology 88:1126-1167.

Jacob, S. W. et al. (1964) Dimethyl sulfoxide (DMSO): a new concept in pharmacotherapy. Current Therapeutic Research 6:134-135.

Johnstone, D. R. and Templeton, M. (1980) The feasibility of palpating the lateral pterygoid muscle. The Journal of Prosthetic Dentistry 44:318-323.

Karaca, M. et al. (1991) Dimethly sulphoxide lowers ICP after closed head trauma. European Journal of Clinical Pharmacology 40:113-114.

Kerr, F. W. L and Olafsson, R. A. (1961) Trigeminal cervical volleys: convergence on single units in the spinal gray at C1 and C2. Archives of Neurology 5:171-178.

Kolb, K. H. et al. (1967) Absorption, distribution, and elimination of labeled dimethyl sulfoxide in man and animals. Annals of the New York Academy of Sciences 141:85-95.

Langmead, L. et al. (2004) Randomized, double-blind, placebo-controlled trial of oral aloe vera gel for active ulcerative colitis. Alimentary Pharmacology and Therapeutics 1;19(7):739-47.

Limmroth, V. et al. (2007) Topiramate in patients with episodic migraine: reducing the risk for chronic forms of headache. Headache 47:13-21.

Lockie, L. M. and Norcross, B. (1967) A clinical study on the effects of dimethyl sulfoxide in 103 patients with acute and chronic musculoskeletal injures and inflammation. Annals of the New York Academy of Sciences 141:599-602.

Matsumoto, J. (1967) Clinical trials of dimethyl sulfoxide in rheumatoid arthritis patients in Japan. Annals of the New York Academy of Sciences 141:560-568.

Moskowitz, M.A. (1992) Interpreting vessel diameter changes in vascular headache. Cephalalgia 12:5-7.

Muir, M. (1996) DMSO: many uses, much controversy. Alternative and Complementary Therapies pp230-235

Nelson, A. J. and Friedman, M. H. (2001) Somatosensory trigeminal evoked potential amplitudes following low level and sham irradiation over time. Laser Therapy 14:60-64.

Nobel, S. L. and Moore, K. L. (1997) Drug treatment of migraine: part II. Preventive therapy. American Family Physician 56(9):2279-2286.

Pugh, N. et al. (2001) Characterization of Aloeride, a new high-molecular-weight polysaccharide from Aloe vera with potent immunostimulatory activity. Journal of Agricultural and Food Chemistry 49(2):1030-4.

Reuter, J. et al. (2008) Investigation of the anti-inflammatory potential of Aloe vera gel (97.5%) in the ultraviolet erythema test. Skin Pharmacology and Physiology 21(2):106-110.

Rapoport, A. et al. (2006) Long-term migraine prevention with topiramate: open-label extension of pivotal trials. Headache, 46:1151-1160.

Ro, J. Y. et al., (2000) Inhibitory mechanism of aloe single component (alprogen) on mediator release in guinea pig lung mast cells activated with specific antigen-antibody reactions. Journal of Pharmacology and Experimental Therapeutics 292(1): 114-121.

Salim, A. S. (1992) Role of oxygen-derived free radical scavengers in the management of recurrent attacks of ulcerative colitis: A new approach. Journal of Laboratory and Clinical Medicine 119:740-747.

Saito, H. et al. (1982) Pharmacological studies on a plant lectin aloctin A. II Inhibitory effect of aloctin A on experimental models of inflammation in rats. The Japanese Journal of Pharmacology 32(1):139-42.

Sherrington, C. S. (1898) Decerebrate rigidity and reflex coordination of movement. The Journal of Physiology 22:319-332.

Shirley, S. W. et al. (1978) Dimethyl sulfoxide in treatment of inflammatory genitourinary disorders. Urology 11:215-220.

Stewart, B. H. et al. (1968) The use of dimethyl sulfoxide (DMSO) in the treatment of interstitial cystitis. The Journal of Urology 98:671-672.

Stewart, B. H. et al. (1972) The treatment of patients with interstitial cystitis, with special reference to intravesical DMSO. The Journal of Urology 107(3):337-380.

Storey, J. R. et al. (2001) Topiramate in migraine prevention: a double-blind placebo-controlled study. Headache, 41:968-975.

Suckert, V. R. (1969) Die Wirkung von Dimethylsulfozyd auf die Crontronol-arthritis des Kaninchen-kniegelenkes. Buchbesprechungen. 81:157-158.

t'Hart, L. A. et al. (1989) An anti-complementary polysaccharide with immunological adjuvant activity from the leaf parenchyma gel of Aloe vera. Planta Medica 55(6):509-12.

Udupa, S. I. et al. (1994) Anti-inflammatory and wound healing properties of Aloe vera. Fitoterapia 65:141-145.

Vazquez, B. et al. (1996) Anti-inflammatory activity of extracts from Aloe vera gel. Journal of Ethnopharmacology 55(1):69-75.

Winner, P. et al. (2005) Topiramate for migraine prevention in children: a randomized double-blind, placebo-controlled trial. Headache, 45:1304-1312.

Yagi, A. et al. (1982) Cardiac stimulant action of constituents of Aloe saponaria. Journal of Pharmaceutical Sciences 71(7):739-41.

I claim:

1. A trigeminal nerve and extended trigeminal nervous system specific, topical anti-inflammatory and analgesic invention, which by a specific application, to areas which correlate to or relate to the various trigeminal branches and plexus, mitigates and acts to prevent migraine-type headache, cluster-type headache, tension-type headache, post-traumatic headache, atypical facial pain and cervical muscle spasm as well as other ideopathic head or facial pain or discomfort, associated with, or exacerbated by, inflammation but without the use of NSAIDS, glucocorticoid steroids or salicylites, said inflammation can be localized and corollary to the maxillary third molar apical area of the trigeminal nerve branches, system and plexus, or generally, in other areas correlated with or related to the intraoral and extraoral trigeminal nerve areas systems or plexus, where pain mitigation action is desirable, to which the invention is topically applied directly to any or all of the following, the gingival area of the mouth in the maxillary third molar apical area correlating to the plexus formed by the posterior superior alveolar branch of the ipsilateral maxillary nerve, the area of the inferior alveolar branch along the lower jaws outer gingival tissue between teeth and cheek encompassing about the area of the rearmost two molars and the wisdom tooth, the extraoral trigeminal nerve areas specified as follows, correlating with, or related to, the location of the, auriculotemporal nerve at the front base of the ear, temporomalar nerve at both jaw and temple, supratrocheal, lacrimal and supraorbital nerves of the forehead, buccal nerve of the cheek, mental nerve of the lower jaw, infratrochlear and nasal nerves along the nose, and infraorbital nerve of the cheek area adjacent the nose and under the eye, the invention is comprised of, in any of its forms, solid, powders, acids, oils, extracts, liquids, gels, sprays, any single or combination of anti-inflammatory herbs or botanicals (initially boswellia subsequently aloe) in any amount (initially to maximum solubility), and mixed into, by any means of combining these, a base vehicle which in this case, said base being comprised of, dimethyl sulfoxide, itself an anti-inflammatory and analgesic substance, in a liquid, gel, solid or semi-solid form, in any concentration between 0.01% and 99.99%, (initially 50% concentration), combined with water and urea or ethanol in any amount (initially water and urea), combined with or without any salts in any amount (initially 0.50%), such as NaCI or KCI, (initially NaCI), mixed by any method of mixing or combining with any, liquids, creams, acids, oils, emulsifiers, thickeners or stabilizers in any amount (initially carbopol subsequently xanthan gum and glycerol), with natural or artificial flavorings in any amount, (initially Mentha x piperita) , with natural or artificial sweeteners (initially stevia), in any amount, none of which are honey due to the risk of the spores of the bacterium clostridium botulinum which is often found in honey, with or without thickeners, solids or emulsifiers as needed in any amount (initially carbopol subsequently xanthan gum and glycerol), to create a liquid, gel, cream, powder, semi-solid or solid, allowing application to and adherence to gingival tissue, and generally, and to facilitate anti-inflammatory and analgesic action upon the tissues on, about, relating to, correlating with, or otherwise surrounding the afore-described areas which in any way relate to the trigeminal nerve and its branches, plexus or structures and is used to affix said anti-inflammatory composition, to, onto, into, beneath or through the gingival tissues and to said areas of inflammation by the normal solvent action of dimethyl sulfoxide, this topical application can be used as needed to mitigate acute symptoms, or as a prophylactic application, to manage chronic symptoms or mitigate the onset of acute symptoms and in order to manage chronic conditions, should the afore-described ingredients or components or any aspects of the invention, now or at some future time, be re-categorized by any government or law-making body, or be subject to any regulatory changes, this is envisioned by this claim and said claim would, therefore, survive any such regulatory changes, the invention considers any variation of the above described, to be inclusive to the invention, under the revisions and modifications statement included in this application, which states, it will be apparent to those well-acquainted in the art that revisions and modifications may be made without departing from my invention in its broader aspects, application or intent.
